(19) United States
(12) Patent Application Publication
(10) Pub. No.: US 2007/0134288 A1
(43) Pub. Date: Jun. 14, 2007

(54) Anti-adhesion agents for drug coatings

(76) Inventors: Edward Parsonage, St. Paul, MN (US); James Lasch, Oakdale, MN (US); Steve Kangas, Woodbury, MN (US); Jan D. Seppala, Greenfield, MN (US)

Correspondence Address: JONES DAY, 222 EAST 41ST ST, NEW YORK, NY, 10017, US

(21) Appl. No.: 11/300,561

(22) Filed: Dec. 13, 2005

(51) Int. Cl. A61F 2/02

(52) U.S. Cl. 424/423

(57) ABSTRACT

Coated medical devices and methods for coating such devices are disclosed. The invention is directed to the use of an anti-adhesion agent in a coating for a medical device. More particularly, the invention is directed to a medical device comprising an anti-adhesion agent that prevents the self-adhesion of different portions of a coating disposed on the surface of the medical device. Additionally, this invention is directed to methods for coating such a medical device.

### FIELD OF THE INVENTION

The invention relates generally to a coating for a medical device containing at least one anti-adhesion agent. More particularly, the invention is directed to a medical device coating comprising an anti-adhesion agent that prevents the adhesion between two surfaces of a medical device of which at least one surface has a coating disposed thereon. Additionally, this invention is directed to methods for making such a medical device coating.

### BACKGROUND OF THE INVENTION

A variety of medical conditions are commonly treated by introducing an insertable or implantable medical device in to the body. In many instances, the medical device is coated with a material, such as a polymer, which is able to release a biologically active agent. For example, various types of drug-coated stents have been used for localized delivery of drugs to a body lumen. See, e.g., U.S. Pat. No. 6,099,562 to Ding et al.

Exposure to a medical device which is implanted or inserted into the body of a patient can cause the body tissue to exhibit adverse physiological reactions. For instance, the insertion or implantation of certain catheters or stents can lead to the formation of emboli or clots in blood vessels. Similarly, the implantation of urinary catheters can cause infections, particularly in the urinary tract. Other adverse reactions to medical devices include cell proliferation which can lead to hyperplasia, occlusion of blood vessels, platelet aggregation, rejection of artificial organs, and calcification.

A medical device can be used not only for reducing such adverse effects, but also for direct administration of a biologically active material into a particular part of the body when a disease is localized to the particular part, such as, without limitation, a body lumen including a blood vessel, for the treatment of the disease. Such direct administration may be more preferred than systemic administration. Systemic administration requires larger amounts and/or different concentrations of the biologically active materials because of indirect delivery of such materials to the afflicted area. Also, systemic administration may cause side effects which may not be a problem when the biologically active material is locally administered.

To reduce the above-mentioned adverse effects and/or to directly administer a biologically active material, pharmaceuticals have been applied to medical devices by covering the surface with a coating containing them. For example, U.S. Pat. Nos. 5,464,650; 5,624,411; and 6,099,562 disclose stents or medical devices having a coating containing a therapeutic substance.

Medical device coating formulations can comprise a polymeric coating such as a polymeric material with elastomeric properties. Elastomeric properties of the polymer coating are often desirable to minimize cracking and provide a more mobile matrix for diffusion release of the drug. Elastomeric coatings also exhibit desired biocompatibility and anti-thrombogenicity properties. However, complications can arise from a tacky polymeric coating including ones with elastomeric properties, as a result of adhesion of coated portions of the device. See e.g., U.S. Pat. No. 5,741,331.

For example, in balloon expandable stents, coating adhesion can result in the formation of webs between struts when the stent is expanded. In particular, when a coated stent is crimped on a balloon, the coated surfaces of the stent can contact each other. The coating on one strut can adhere to the coating on another strut. When the balloon is expanded to deploy the stent, the adhered coating can form webs between the struts. These webs can result in coating damage such as fracture and delamination from the stent substrate. Additional problems can arise with self-expanding stents, where adhesion of the coating can significantly reduce the deployment force of the stent when the sheath is removed to allow expansion of the stent. This could result in insufficient deployment of the stent and subsequent embolization. Furthermore, adhesion of the coating on an implantable device to the delivery system may cause procedural problems during implantation. It would thus be desirable in certain instances to reduce the adhesive properties of the coating on a medical device such as a stent.

A new and non-obvious means for reducing the adhesive properties of a coating is the use of an anti-adhesion agent in the coating. The anti-adhesion agent prevents the coated surfaces from intimate contact and/or prevents adhesion.

### SUMMARY OF THE INVENTION

In one embodiment, the present invention is directed to an implantable medical device comprising a surface and a coating disposed on at least a part of the surface. The coating comprises a biologically active material, a first polymeric material, and a chemical anti-adhesion agent. The chemical anti-adhesion agent reduces, e.g., lowers or prevents the adhesion or tack of the coating, as compared to the same coating without the chemical anti-adhesion agent. The chemical anti-adhesion agent can dispersed in the coating. Also, in some embodiments the coating can have an outer surface and the concentration of the chemical anti-adhesion agent is different at the outer surface than the concentration of the chemical anti-adhesion agent within the coating, e.g., the concentration at the outer surface is higher.

Furthermore, in certain embodiments, the coating comprises an under layer and a top layer which is disposed over the under layer. The top layer comprises the chemical anti-adhesion agent. Also, the top layer can further comprise a second polymeric material. Moreover, the under layer can comprise the biologically active material and the first polymeric material.

In general the chemical anti-adhesion agent will be biocompatible for the intended use. Furthermore, the chemical anti-adhesion agent may be biostable, bioabsorbable, or biodegradable. The chemical anti-adhesion agent can be water soluble. Additionally, the chemical anti-adhesion agent can comprise a nonionic surfactant. Examples of nonionic surfactants would include, but are not limited to, a C₁₂-C₂₄ fatty acid; a C₁₈-C₃₆ mono-,di-and triacylglyceride; a sucrose fatty acid ester; a sorbitan fatty acid ester; a C₁₆-C₁₈ fatty alcohol; an ester of a fatty alcohol or fatty acid; an anhydride of a fatty acid; metallic complexes of fatty acids, and organo-onium compounds, to name a few. The chemical anti-adhesion agent can compromise a biosurfactant such as an ionizable biosurfactant. Also, the chemical anti-adhesion agent can comprise an ionic surfactant such as a lauryl sulfate or phosphatidyl choline. Furthermore, the chemical anti-adhesion agent can comprise a surface active low molecular weight compound, medium molecular weight oligomer or high molecular weight polymer, such as a silicone or a fluorinated ether.

In one embodiment, the biologically active material can comprise paclitaxel. In another embodiment, the biologically active material can comprise rapamycin, i.e., sirolimus, tacrolimus, everolimus, ABT578, or other limus derivatives.

Moreover, in addition to the chemical anti-adhesion agent, the coating can further comprise a physical anti-adhesion agent. The concentration of the physical adhesion agent can be different at the outer surface of the coating than the concentration of the physical adhesion agent within the coating. Examples of suitable physical anti-adhesion agents include, without limitation, solid glass spheres, glass bubbles, other mineral, or polymeric particles.

In another embodiment, the invention is directed to an implantable medical device comprising a surface and a coating disposed on at least a part of the surface in which the coating comprises a biologically active material, a first polymeric material, and a physical anti-adhesion agent. The physical anti-adhesion agent reduces, e.g., lowers or prevents, the adhesion or tack of the coating as compared to the same coating without the physical anti-adhesion agent. The physical anti-adhesion agent can be dispersed in the coating. The coating can have an outer surface and the concentration of the physical anti-adhesion agent can be different, i.e., higher, at the outer surface than the concentration of the physical anti-adhesion agent within the coating. In certain embodiments, the coating comprises an under layer and a top layer which is disposed over the under layer, and the top layer comprises physical anti-adhesion agent. The top layer can further comprise a second polymeric material. The under layer can comprise the biologically active material and the first polymeric material.

Also, in some embodiments, the physical anti-adhesion agent can comprise an organic material. The organic material can comprise at least one cross-linked polymeric sphere or organic aggregate. The physical anti-adhesion agent can comprise an inorganic material. In some embodiment, the physical anti-adhesion agent can comprise at least solid glass spheres, glass bubbles, or mineral particles. The at least one mineral particle can comprise calcium carbonate or talc. Furthermore, in some embodiments, the biologically active material comprises paclitaxel. Also, the biologically active material can comprise rapamycin, i.e., sirolimus, tacrolimus, everolimus, ABT578, or other limus derivatives and combinations thereof.

In another embodiment, the invention is directed to a stent comprising a surface and a coating disposed on at least a part of the surface in which the coating comprises a biologically active material, a first polymeric material, and a chemical anti-adhesion agent comprising a nonionic surfactant. The chemical anti-adhesion agent reduces, e.g., lowers or prevents the adhesion or tack of the coating, as compared to the same coating without the chemical anti-adhesion agent. The nonionic surfactant can be dispersed in the coating. Also, the coating can have an outer surface and the concentration of the nonionic surfactant can be different, e.g., higher at the outer surface than the concentration of the nonionic surfactant within the coating.

In another embodiment, the invention is directed to a stent comprising a surface and a coating disposed on at least a part of the surface in which the coating comprises as biologically active material, a first polymeric material, and a physical anti-adhesion agent. The physical anti-adhesion agent reduces, e.g., lowers or prevents, the adhesion or tack of the coating as compared to the same coating without the physical anti-adhesion agent. The physical anti-adhesion agent can be dispersed in the coating. Also, the coating can have an outer surface and the concentration of the physical anti-adhesion agent can be different, e.g., higher at the outer surface than the concentration of the physical anti-adhesion agent within the coating.

### BRIEF DESCRIPTION OF THE DRAWINGS

FIG. 1A represents an embodiment where the surface of a medical device is coated with coating comprising a polymer, a chemical anti-adhesion agent, and a biologically active material.

FIG. 1B represents an embodiment where the surface of a medical device is coated with a coating comprising a different concentration of a chemical anti-adhesion agent at the outer surface of the coating than dispersed in the coating.

FIG. 1C represents an embodiment where the surface of a medical device is coated with a coating comprising an under layer, which comprises a polymer and a biologically active material, and a top layer, which comprises a chemical anti-adhesion agent, disposed over the under layer.

FIG. 1D represents an embodiment where the surface of a medical device is coated with a coating comprising an under layer, which comprises a polymer and a biologically active material, and a top layer, which comprises a chemical anti-adhesion agent dispersed in a polymer.

FIG. 2A represents an embodiment where the surface of a medical device is coated with a coating comprising a polymer, a physical anti-adhesion agent, and a biologically active material.

FIG. 2B represents an embodiment where the surface of a medical device is coated with a coating comprising a different concentration of a physical anti-adhesion agent at the outer surface of the coating than disposed in the coating.

FIG. 2C represents an embodiment where the surface of a medical device is coated with a coating comprising an under layer, which comprises a polymer and a biologically active material, and a top layer, which comprises a physical anti-adhesion agent and a polymer.

FIG. 3 represents two portions of a medical device that have been coated with a coating containing anti-adhesion agents, in which the portions contact each other.

### DETAILED DESCRIPTION OF THE INVENTION

### 1. Embodiments Comprising a Chemical Anti-Adhesion Agent

The implantable medical device of the present invention has a surface and a coating disposed on at least a part of the surface. In one embodiment, the coating comprises a biologically active material, a polymeric material, and a chemical anti-adhesion agent. The term “chemical anti-adhesion agent” refers to a chemical that forms a barrier on a surface of a coating on a medical device and through the absence of cohesive strength and/or weak boundary layers, reduces, e.g., lowers or prevents, adhesion of that surface of the coating to a material such as, but not limited to, another portion of the coating or an uncoated portion of the medical device. The amount of adhesion reduced can be measured as a reduction in tack force.

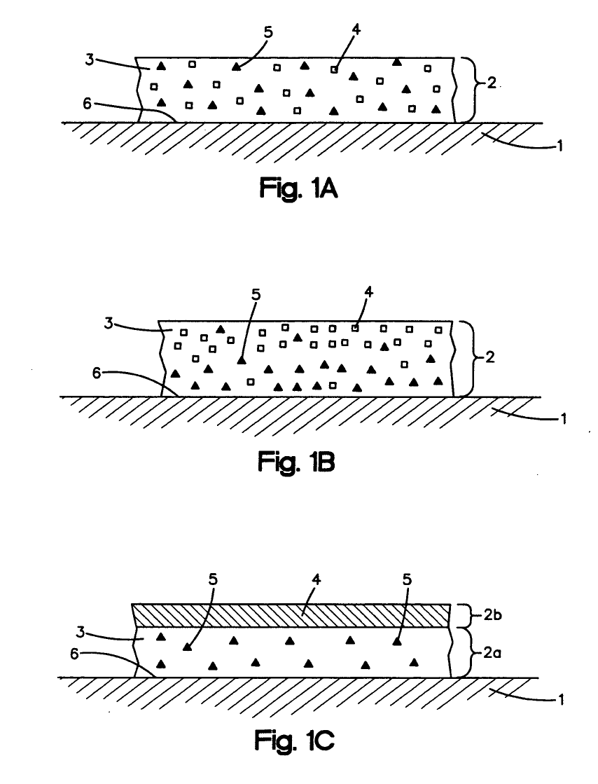

FIGS. 1A-1D are cutaway side views of various embodiments of the present invention comprising a chemical anti-adhesion agent. FIG. 1A illustrates an embodiment where the surface 6 of a medical device 1 is coated with a coating 2 comprising a polymer 3, a chemical anti-adhesion agent 4, and a biologically active material 5. In this embodiment, the chemical anti-adhesion agent 4 and the biologically active material 5 are dispersed in the polymer 3, which is disposed on the surface 6 of the medical device 1. Because many of the chemical anti-adhesion agents have a low surface energy, their concentration at the outer surface of a coating may be different than that within the coating. FIG. 1B shows such an embodiment, where more of the chemical anti-adhesion agent 4 is concentrated at the outer surface of the coating 2. In some embodiments most of the chemical anti-adhesion agent can be at the outer surface. For example, at least 80% of the chemical anti-adhesion agent in the coating can be at the outer surface. In other embodiments, the chemical anti-adhesion agent is less concentrated at the outer surface of the coating 2 than in other parts of the coating.

FIG. 1C represents an embodiment where the surface 6 of a medical device 1 is coated with a coating comprising an under layer 2a, which comprises a polymer 3 and a biologically active material 5, and a top layer 2b, which comprises a chemical anti-adhesion agent 4 disposed over the under layer 2a. In some embodiments, the top layer 2b can be a protective water soluble layer that acts as a temporary anti-adhesion layer. This layer can cover parts of or the entire under layer 2a. Over time the layer will dissolve away in the blood. Examples of materials suitable for forming a temporary or dissolvable anti-adhesion layer includes without limitation water soluble polymers such as polyvinyl alcohol, polyvinyl pyrrolidone (PVP), polyethylene oxide and biological-based materials such as sodium heparin.

FIG. 1D illustrates an embodiment where the surface 6 of a medical device 1 is coated with a coating comprising an under layer 2a. The under layer 2a comprises a polymer 3 in which a biologically active material 5 is dispersed. This embodiment also includes a top layer 7 which is disposed over the under layer 2a. The top layer 7 comprises a dispersion of the chemical anti-adhesion agent 4 in a polymer 8 for preventing adhesion of the coating to material such as another coated portion of the medical device. Although FIGS. 1C and 1D show the top layer 2b or 7 disposed directly over the under layer 2a, in certain embodiments, there can be intervening coating layers between the top layer and the underlayer.

In certain embodiments, more than one chemical anti-adhesion can be used. Each of the different chemical anti-adhesion agents can cover or be incorporated into some or all parts of the coating.

Suitable chemical anti-adhesion agents include any surface active compositions which reduces the surface tack of the coating. These agents may be known polymeric anti-adhesion agents such as silicones and fluorine containing polymers, for example. These agents may also consist of known biosorbable and biodegradable compositions which act to reduce the surface adhesive properties. These agents may further include intermediate molecular weight compounds such as oligomers of polyethers and alkanes, or biological oils such as fatty esters, to name a few. These agents may also be low molecular weight surface active compounds such as low molecular weight silicones, fluorinated materials, or biological compounds such as sugars.

Chemical anti-adhesion agents may further include various surfactant compositions. These surfactant agents may be nonionic or ionic in composition. Nonionic surfactants are defined as those agents which are amphiphilic in nature but do not readily ionize in aqueous solution. Nonionic surfactants may include, for example C₁₂-C₂₄ fatty acids such as lauric acid, myristic acid, palmitic acid, stearic acid, arachidic acid, behenic acid, and lignoceric acid; C₁₈-C₃₆ mono-, di-and triacylglycerides such as glyceryl monooleate, glyceryl monolinoleate, clyceryl monolaurate, glyceryl mondocosanoate, glyceryl monomyristate, glyceryl monodicenoate, glyceryl dipalmitate, glyeryl didocosanoate, glyceryl dimyristate, glyceryl didecenoate, glyceryl tridocosanoate, glyceryl trimyristate, glyceryl tridecenoate, glycerol tristearate and mixtures thereof, sucrose fatty acid esters such as sucrose distearate and sucrose palmitate; sorbitan fatty acid esters such as sorbitan monostearate, sorbitan monopalmitate and sorbitan tristerate; C₁₆-C₁₈ fatty alcohols such as cetyl alcohol, myristyl alcohol, stearyl alcohol, and cetostearyl alcohol, esters of fatty alcohols or fatty acids such as cetyl palmitate and cetearyl palmitate; anhydrides of fatty acids such as stearic anhydride. Nonionic surfactants may further include various metallic salts, such as calcium stearate, magnesium stearate, and zinc stearate, to name a few. Nonionic surfactants may also include organo-onium compounds. Ionic surfactants are defined as those agents which are polar in nature and readily ionize in solution. Ionic surfactants would generally include organic compounds containing salts of strong acid and bases. Examples of ionic surfactants would include, for example, lauryl sulfates such as ammonium lauryl sulfate. Ionic surfactants may further include certain biological lipids, such as phosphatidyl coline.

The chemical anti-adhesion agent can be present in an amount of about 0.0001 to about 99 weight percent of the coating or coating layer in which the chemical anti-adhesion is contained. If the chemical anti-adhesion agent is in the top layer, the chemical anti-adhesion agent can be >99 weight percent of the top layer. Preferably, the chemical anti-adhesion agent is about 0.001 to 90 weight percent of the coating or coating layer in which the chemical adhesion agent is contained. In some embodiments the nonionic surfactant can be present in an amount of about 0.001 to about 50 weight percent of the coating or coating layer in which the chemical anti-adhesion agent is contained. More preferably, the nonionic surfactant is present in an amount of 0.001 to 1 weight percent of the coating or coating layer in which the chemical anti-adhesion agent is contained. In some embodiments the ionic surfactant can be present in an amount of about 0.001 to about 50 weight percent of the coating or coating layer in which the chemical anti-adhesion agent is contained. More preferably, the ionic surfactant can be present in an amount of 0.001 to 1 weight percent of the coating or coating layer in which the chemical anti-adhesion agent is contained.

The chemical anti-adhesion agent can reduce the tack force of the coating by about 5 to about 99 %, depending on the loading. In some embodiments, the task force of the coating can be reduced by about 5 to about 95% or about 10 to about 75%, depending on the load.

### 2. Embodiments Comprising a Physical Anti-Adhesion Agent

Another aspect of the present invention includes a medical device with a surface and a coating disposed on at least a part of the surface, wherein the coating comprises a biologically active material, a polymeric material, and a physical anti-adhesion agent. The term “physical anti-adhesion agent” refers to a rigid material which acts as a barrier on a surface of a coating on a medical device to reduce, e.g., lower or prevent, adherence of that surface of the coating to a material such as but not limited to another portion of the coating or an uncoated portion of the medical device. The amount of adhesion reduced can be measured as a reduction in tack force.

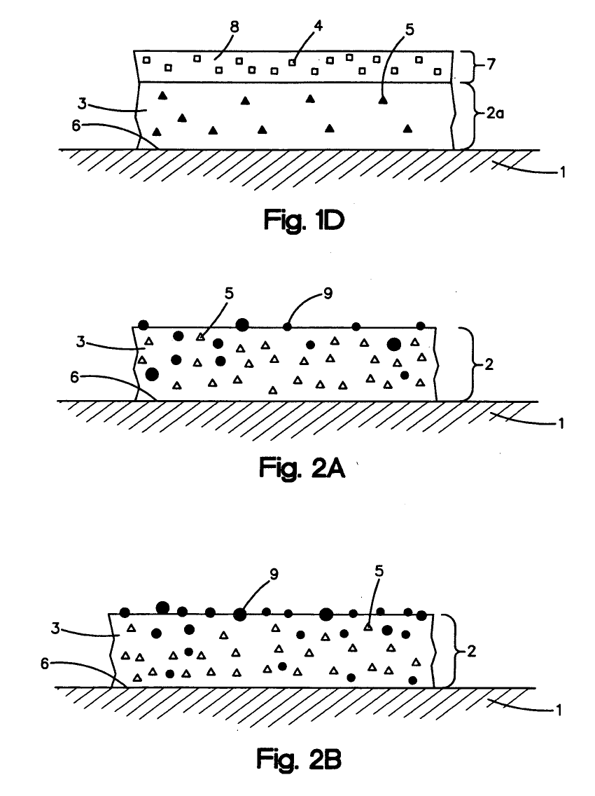

FIGS. 2A-2C are cutaway side views of various embodiments of the present invention comprising a physical anti-adhesion agent. FIG. 2A represents an embodiment where the surface 6 of a medical device 1 is coated with a coating 2 comprising a polymer 3, a physical anti-adhesion agent 9, and a biologically active material 5. The biologically active material 5 and the physical anti-adhesion agent 9 are dispersed in the polymer coating. Preferably, at least a portion of the physical anti-adhesion agent 9 protrudes from the polymer 3 in order to prevent contact between polymer 3 and other materials such as polymer coated surfaces.

FIG. 2B illustrates an embodiment where the surface 6 of a medical device 1 is coated with a coating 2 comprising a biologically active material 5 dispersed in a polymer 3 and a physical anti-adhesion agent 9 concentrated at the outer surface of the coating 2. As shown in this figure, the physical anti-adhesion agent 9 can be disposed on the outer surface of the coating 2. In some embodiments, most of the physical anti-adhesion agent can be at the outer surface. For instance, at least 80% of the physical anti-adhesion agent in the coating can be at the outer surface. Alternatively, the concentration of the physical anti-adhesion agent is less at the outer surface of the coating than that in other parts of the coating.

FIG. 2C represents an embodiment where the surface 6 of a medical device 1 is coated with a coating. The coating has an under layer 2a comprising a polymer 3 in which a biologically active material 5 is dispersed. This embodiment also includes a top layer 7 which is disposed on the under layer 2a. The top layer 7 comprises a polymer 8 and a physical anti-adhesion agent 9. Physical anti-adhesion agent 9 protrudes from top layer 7 in order to prevent contact between top layer 7 and other materials such as the polymer coated surfaces. Although FIG. 2C shows the top layer 7 disposed directly over the under layer 2a, in certain embodiments, there can be intervening coating layers between the top layer and the under layer.

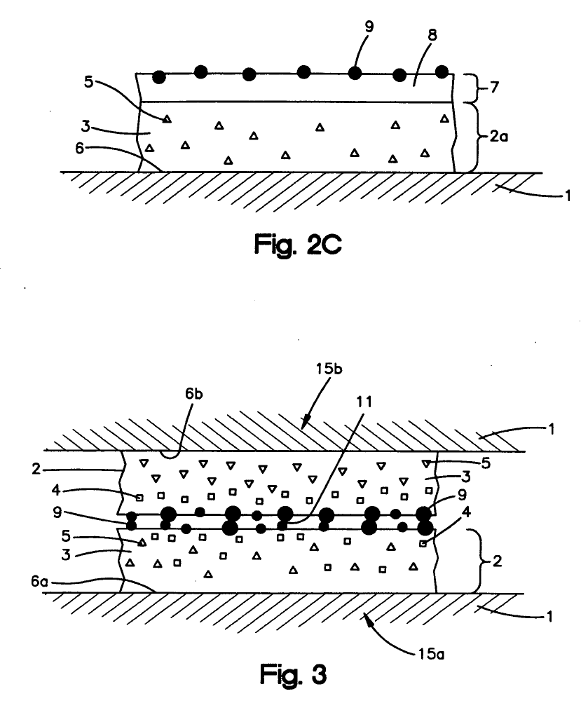

FIG. 3 represents two coated surfaces 6a, 6b contacting each other at points 11. The coating comprising a physical anti-adhesion agent 9 as well as a chemical anti-adhesion agent 4. Each portion 15a, 15b of the medical device 1 comprises a surface 6a, 6b which has been coated with a coating 2 comprising a polymer 3. In this embodiment, biologically active material 5 and chemical anti-adhesion agent 4 are dispersed in the polymer 3. Physical anti-adhesion agent 9 is also dispersed in the polymer 3 but is concentrated near the outer surface of the coating 2. At least a portion of the physical anti-adhesion agent 9 protrudes from polymer 3 so that the physical anti-adhesion agent 9, which is dispersed in a coating of a first portion 15a of the medical device, can contact another physical anti-adhesion agent 9, which is dispersed in a coating of a second portion 15b of the medical device, at points 11. The ability for the physical anti-adhesion agents to contact each other at points 11 reduces contact between the coating of first and second portions 15a, 15b of the medical device 1.

In certain embodiments, more than one physical anti-adhesion agent can be used. Each of the different physical anti-adhesion agents can cover or be incorporated into some or all parts of the coating.

Examples of physical anti-adhesion agents include organic or inorganic materials. Examples of organic physical anti-adhesion agents include, without limitation, polymeric spheres and organic aggregates or a thin, rigid polymeric layer. Inorganic physical anti-adhesion agents include, without limitation, solid glass spheres, glass bubbles, and mineral particles such as calcium carbonate and talc.

The physical anti-adhesion agent can be present in an amount of about 0.0001 to about 99 weight percent of the coating or coating layer in which the physical anti-adhesion is contained. If the physical anti-adhesion agent is in the top layer, the physical anti-adhesion agent can be >99 weight percent of the top layer. Preferably, the physical anti-adhesion agent can be present in an amount of about 0.001 to about 90 weight percent of coating or coating layer in which the physical anti-adhesion is contained. Also, the physical anti-adhesion agent can be present in an amount of about 0.001 to about 50 weight percent or preferably 0.01 to about 25 weight percent of coating layer in which the physical anti-adhesion is contained.

Also, the physical anti-adhesion agent can cover about 0.1% to about 100% of the outer surface area of the coating or coating layer. Preferably, the physical anti-adhesion agent covers about 50% to about 95% of the outer surface area of the coating or coating layer.

The physical anti-adhesion agent can reduce the tack force of the coating by 5 to about 95 %, depending on the loading.

### 3. Suitable Polymers for the Coatings

The polymer(s) useful for forming the coating the medical device should be one(s) that is biocompatible and avoid irritation to body tissue. It can be either biostable or bioabsorbable. Suitable polymeric materials include, without limitation, cross-linked elastomers such as silicones elastomers (e.g., polysiloxanes and substituted polysiloxanes) and EPDM rubbers, thermoplastic elastomers such as polyurethanes, and thermoplastics such as ethylene vinyl acetate copolymers and polyacrylates and biodegradable polyesters, and various polyolefin elastomers.

Suitable polymeric materials used in the coating compositions of the present invention can also include without limitation: polyurethanes, silicones (e.g., polysiloxanes and substituted polysiloxanes), polyesters, styrene-isobutylene copolymers, polymers that can be dissolved and cured or polymerized on the medical device or polymers having relatively low melting points that can be blended with biologically active materials, thermoplastic elastomers, polyolefins, polyisobutylene, ethylene-alphaolefin copolymers, acrylic and acrylates and phosphatidyl coline based copolymers, acrylate polymers, and copolymers, vinyl halide polymers and copolymers such as poly(lactide-co-glycolide) (PLGA), polyvinyl alcohol (PVA), poly(L-lactide) (PLLA), polyanhydrides, polyphosphazenes, polycaprolactone (PCL), polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate. copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid (PLA), polyglycolic acid (PGA), polyethylene oxide (PEO), polylactic acid-polyethylene oxide copolymers, EPDM (etylene-propylene-diene) rubbers, fluorosilicones, polyethylene glycol (PEG), polyalkylene glycol (PAG), polysaccharides, phospholipids, and combinations of the foregoing.

In certain embodiments, the polymeric material is hydrophilic (e.g., PVA, PLLA, PLGA, PEG, and PAG). In certain other embodiments, the polymeric material is not hydrophilic (e.g., PLA, PGA, polyanhydrides, polyphosphazenes and PCL). In yet other embodiments, the polymeric material is hydrophobic (e.g., polyolefins and fluoropolymers).

More preferably for medical devices which undergo mechanical challenges, e.g., expansion and contraction, the polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition does not possess a distinct drop in load after the yield point when the device is subjected to forces, stress or mechanical challenge.

In some embodiments, the polymeric materials are biodegradable. Biodegradable polymeric materials can degrade as a result of hydrolysis of the polymer chains into biologically acceptable, and progressively smaller compounds. In one embodiment, a polymeric material comprises polylactides, polyglycolides, or their co-polymers. Polylactides, polyglycolides, and their co-polymers break down to lactic acid and glycolic acid, which enters the Kreb's cycle and are further broken down into carbon dioxide and water.

The polymeric materials can also degrade through bulk hydrolysis, in which the polymer degrades in a fairly uniform manner throughout the matrix. For some novel degradable polymers, most notably the polyanhydrides and polyorthoesters, the degradation occurs only at the surface of the polymer, resulting in a release rate that is proportional to the surface area of the drug therapeutic agents and/or polymer/therapeutic agent mixtures. Hydrophilic polymeric materials such as PLGA will erode in a bulk fashion. Various commercially available PLGA may be used in the preparation of the coating compositions. For example, poly(d,1-lactic-co-glycolic acid) are commercially available. A preferred commercially available product is a 50:50 poly(d,1-lactic-co-glycolic acid) (d,1-PLA) having a mole percent composition of 50% lactide and 50% glycolide. Other suitable commercially available products are 65:35, 75:25, and 85:15 poly(d,1-lactic-co-glycolic acid). For example, poly(lactide-co-glycolides) are also commercially available from Boehringer Ingelheim (Germany) under the trade name Resomerg, e.g., PLGA 50:50 (Resomer RG 502), PLGA 75:25 (Resomer RG 752) and d,1-PLA (resomer RG 206), and from Birmingham Polymers (Birmingham, Alabama). These copolymers are available in a wide range of molecular weights and ratios of lactic to glycolic acid.

In one embodiment, the coating comprises copolymers with desirable hydrophilic/hydrophobic interactions (see, e.g., U.S. Pat. No. 6,007,845, which describes nanoparticles and microparticles of non-linear hydrophilic-hydrophobic multiblock copolymers, which is incorporated by reference herein in its entirety). In a specific embodiment, the coating comprises ABA triblock copolymers consisting of biodegradable A blocks from PLG and hydrophilic B blocks from PEO.

### 4. Suitable Biologically Active Materials

The term “biologically active material” encompasses therapeutic agents, and also genetic materials and biological materials. The biologically active materials named herein include their analogs and derivatives. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, paclitaxel (as well as its derivatives, analogs or paclitaxel bound to proteins, e.g. Abraxane™) 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell fumction and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifinctional molecules consisting of a growth factor and a cytotoxin, bifuinctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolide agents such as sirolimus, pimerolimus, or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl) glutamide hydrochloride salt.

Other suitable therapeutic agents include tacrolimus; halofuiginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

Methods suitable for applying biologically active materials to the devices of the present invention preferably do not alter or adversely impact the therapeutic properties of the biologically active material.

### 5. Suitable Medical Devices

The coated medical devices of the present invention can be inserted and/or implanted in the body of a patient. Medical devices suitable for the present invention include, but are not limited to, stents, surgical staples, catheters, such as balloon catheters, central venous catheters, and arterial catheters, guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, septal defect devices, hemodialysis units, hemoperfusion units and plasmapheresis units.

Medical devices suitable for the present invention include those that have a tubular or cylindrical-like portion. The tubular portion of the medical device need not be completely cylindrical. For instance, the cross-section of the tubular portion can be any shape, such as rectangle, a triangle, etc., not just a circle. Such devices include, without limitation, stents, balloon catheters, and grafts. A bifurcated stent is also included among the medical devices which can be fabricated by the method of the present invention.

Medical devices that are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. In certain embodiments, the stent comprises an open lattice sidewall stent structure. When such stents are used, it is in some instances preferable to have the coating disposed on the stent to conform to the stent to preserve the open lattice sidewall structure. In preferred embodiments, the stent suitable for the present invention is an Express stent. More preferably, the Express stent is an Express™ stent or an Express2™ stent (Boston Scientific, Inc. Natick, Mass.).

Medical devices that are suitable for the present invention may be fabricated from metallic, ceramic, or polymeric materials, or a combination thereof. Preferably, the materials are biocompatible. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Suitable ceramic materials include, but are not limited to, oxides, carbides, or nitrides of the transition elements such as titanium oxides, hafnium oxides, iridiumoxides, chromium oxides, aluminum oxides, and zirconium oxides. Silicon based materials, such as silica, may also be used. The polymeric material may be biostable. Also, the polymeric material may be biodegradable. Suitable polymeric materials include, but are not limited to, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, and Teflon.

Polymeric materials that may be used for forming the medical device in the present invention include, without limitation, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), polyethylene glycol dimethacrylate, poly(methyl methacrylate), poly(2-hydroxyethyl methacrylate), polytetrafluoroethylene poly(HEMA), polyhydroxyalkanoates, polycarbonate, poly(glycolide-lactide) co-polymer, polylactic acid, poly(y-caprolactone), poly(y-hydroxybutyrate), polydioxanone, poly(y-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like. Suitable elastomeric polyers include polyurethanes, polysiloxanes, poly(dimethyl siloxanes) and polyphosphazenes.

Also preferable as a polymeric material are styrene-isobutylene-styrene copolymers. Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (ethylene-propylene-diene) rubbers, fluorosilicones, polyethylene glycol, polysaccharides, phospholipids, and combinations of the foregoing.

Preferably, for medical devices which undergo mechanical challenges, e.g., expansion and contraction, polymeric materials should be selected from elastomeric polymers such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

### 6. Methods for Making Coatings

A method of making a coated medical device is also presented. This method comprises providing a medical device having a surface and applying a coating composition on at least a part of the surface, wherein the coating composition comprises a biologically active material, a polymeric material, and an anti-adhesion agent. The embodiment in FIGS. 1A, 1B, 2A and 2B can be formed in such a manner.

In one embodiment, the biologically active material is combined with the polymer to form a first coating composition, which is applied to the device surface to form an under layer. A second composition comprising an anti-adhesion agent and a polymer is formed. The second coating composition is applied to form a top layer disposed on the under layer. The embodiments shown in FIGS. 1C, 1D and 2C can be formed in such a manner. Also, in other embodiments, the coating can be formed by applying a composition of biologically active material and polymeric material to form a coating or coating layer. Subsequently, the physical and/or chemical anti-adhesion agent can be disposed over the coating or coating layer of biologically active material and polymeric material. In such embodiments, the anti-adhesion agent can be concentrated at the outer surface of the coating or coating layer.

A solvent can be used to form the coating compositions. Suitable solvents used to prepare coating compositions include ones which can dissolve or suspend the polymeric material in solution. Examples of suitable solvents include, but are not limited to, tetrahydrofuran, methylethylketone, chloroform, toluene, acetone, isooctane, 1,1,1,-trichloroethane, dichloromethane, isopropanol, IPA, and mixture thereof. Solvents that increase the chemical anti-adhesion agent concentration at the surface relative to the bulk concentration may be preferred.

Coating compositions can be applied by any method to a surface of a medical device to form a coating layer. Examples of suitable methods include, but are not limited to, spraying such as by conventional nozzle or ultrasonic nozzle, dipping, rolling, electrostatic deposition, and a batch process such as air suspension, pancoating or ultrasonic mist spraying. Also, more than one coating method can be applied on the surface of the medical device.

A biologically active material may be delivered to a body lumen using the medical devices described above. The stent, or other medical device, is inserted into body of the patient by a method known to artisan. For example, when the stent of the present invention is a self-expandable stent, then the stent is collapsed to a small diameter by placing it in a sheath, introduced into a lumen of a patient's body using a catheter, and is allowed to expand in the target area by removing it from the sheath. When the stent of the present invention is a balloon expandable stent, the stent is collapsed to a small diameter, placed over an angioplasty balloon catheter, and moved into the area to be placed. When the balloon is inflated, the stent expands.

### EXAMPLES

### Example 1

### Coatings with a Chemical Anti-Adhesion Agent

One of the following chemical anti-adhesion agents was added to a 25% solution of styrene-isobutylene copolymer dissolved in THF and toluene. The amount of anti-adhesion agent loading was 2 wt % (based on weight of polymer). Coatings were cast onto PET film using a knife coater to give a dry coating thickness of about 20 μm. The coatings were dried at 80° C. for 1 hour. Tack was measured using a stainless steel probe tip placed in contact with the coating surface with an applied weight of 50 g for 5 seconds. The force (in grams) required to pull the probe from the surface was measured. Tack force results are shown in Table 1:

TABLE 1

| Anti-Adhesion Agent | Agent Type | Tack Force(g) |
|---|---|---|
| None | | 42 |
| Silwet 7087 (Setre Chemical) | Silicone | 23 |
| FC 430 (3M) | Fluorochemical | 27 |

### Example 2

### Coatings with a Chemical Anti-Adhesion Agent

One of the following anti-adhesion agents was used to form a coating (2% solids in water), which was applied to the styrene-isobutylene copolymer coating using a #7 wire-wound coating bar. The coating was dried at 80° C. for 15 minutes. Tack was measured as described in Example 1. Tack force results are shown in Table 2:

TABLE 2

| Anti-Adhesion Agent | Agent Type | Tack Force (g) |
|---|---|---|
| None | | 42 |
| Pluronic 17R4 (BASF) | Nonionic Surfactant | 32 |
| Rhodapon BOS (Rhodia) | Anionic Surfactant | 8 |
| Rhodapon LSB (Rhodia) | Anionic Surfactant | 5 |

### Example 3

### Coatings with Physical Anti-Adhesion Agents

Cross-linked styrene beads or fluorochemical particles were added to a 25% solution of styrene-isobutylene copolymer dissolved in THF (fluorochemical particles) and toluene (cross-linked beads). The fluorochemical particles were added at 2 wt % (based on weight of polymer) and the beads were added at 0.5 wt % (based on weight of polymer). The coatings were applied as described in Example 1. Tack force results are shown in Table 3:

TABLE 3

| Particle | Tack Force (g) |
|---|---|
| None | 42 |
| Teflon (Zonyl MP 1400; Dupont) | 34 |
| 4 μm (dia) Crosslinked Polystyrene bead | 27 |
| (Bang Beads) | |

### Example 4

### Coatings with Dissolvable Anti-Adhesion Layer

Polyvinylpyrrolidone (PVP) (K-15: ISP Inc) was prepared as a 5% solution in methanol. The PVP solution was applied to a styrene-isobutylene copolymer coating using a #7 wire-wound coating bar and dried at 65° C. for 15 minutes. Tack force was measured as described in Example 1. The coating was then rinsed with water for about 30 second to remove the PVP. The coating was then dried and tack was measured. The results show that one can temporarily reduce tack by overcoating the polymer with a hard top layer and that the underlying layer is retained after dissolution of the topcoat.

TABLE 4

| Coating | Tack Force (g) | |
|---|---|---|
| Styrene-isobutylene copolymer | 46 | g |
| Styrene-isobutylene copolymer + PVP | 0.75 | g |
| coating | | |
| Styrene-isobutylene copolymer after | 53 | g |
| removal of PVP coating | | |

While the foregoing description and drawings may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, and proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description. Furthermore, all references mentioned herein are incorporated by reference in their entirety for all purposes.

What is claimed is:

1. An implantable medical device comprising a surface and a coating disposed on at least a part of the surface, wherein the coating comprises a biologically active material, a first polymeric material, and a chemical anti-adhesion agent wherein the chemical anti-adhesion agent reduces the adhesion of the coating as compared to the same coating without the chemical anti-adhesion agent.

2. The medical device of claim 1, wherein the coating has an outer surface and the concentration of the chemical anti-adhesion agent is different at the outer surface than the concentration of the chemical anti-adhesion agent within the coating.

3. The medical device of claim 1, wherein the coating comprises an under layer and a top layer which is disposed over the under layer, wherein the top layer comprises the chemical anti-adhesion agent.

4. The medical device of claim 3, wherein the chemical anti-adhesion agent is bioabsorbable.

5. The medical device of claim 3, wherein the top layer further comprises a second polymeric material.

6. The medical device of claim 3, wherein the under layer comprises the biologically active material and the first polymeric material.

7. The medical device of claim 1, wherein the chemical anti-adhesion agent comprises a nonionic surfactant.

8. The medical device of claim 1, wherein the chemical anti-adhesion agent comprises an ionic surfactant comprising a biosurfactant.

9. The medical device of claim 1, wherein the chemical anti-adhesion agent comprises lauryl sulfate or phosphatidyl coline.

10. The medical device of claim 1, wherein the biologically active material comprises paclitaxel.

11. The medical device of claim 1, wherein the biologically active material comprises rapamycin, sirolimus, tacrolimus, everolimus, ABT578, or other limus derivatives.

12. The medical device of claim 1, further comprising a physical anti-adhesion agent.

13. A stent comprising a surface and a coating disposed on at least a part of the surface, wherein the coating comprises a biologically active material, a first polymeric material, and a chemical anti-adhesion agent, wherein the chemical anti-adhesion agent comprises a nonionic surfactant; and wherein the coating has an outer surface and the concentration of the nonionic surfactant is different at the outer surface than the concentration of the nonionic surfactant within the coating, wherein the chemical anti-adhesion agent reduces the adhesion of the coating as compared to the same coating without the chemical anti-adhesion agent.

14. The stent of claim 13, wherein the concentration of the nonionic surfactant is higher at the outer surface than the concentration of the nonionic surfactant within the coating.

15. An implantable medical device comprising a surface and a coating disposed on at least a part of the surface, wherein the coating comprises a biologically active material, a first polymeric material, and a physical anti-adhesion agent, wherein the physical anti-adhesion agent reduces the adhesion of the coating as compared to the same coating without the physical anti-adhesion agent.

16. The medical device of claim 15, wherein the coating has an outer surface and the concentration of the physical anti-adhesion agent is different at the outer surface than the concentration of the physical anti-adhesion agent within the coating.

17. The medical device of claim 15, wherein the coating comprises an under layer and a top layer which is disposed over the under layer, wherein the top layer comprises the physical anti-adhesion agent.

18. The medical device of claim 17, wherein the top layer further comprises a second polymeric material.

19. The medical device of claim 17, wherein the under layer comprises the biologically active material and the first polymeric material.

20. The medical device of claim 15, wherein the physical anti-adhesion agent comprises at least solid glass spheres, glass bubbles, or mineral particles.

21. The medical device of claim 15, wherein the biologically active material comprises paclitaxel.

22. The medical device of claim 15, wherein the biologically active material comprises rapamycin, sirolimus, tacrolimus, everolimus, or ABT578, or other limus derivatives.

23. A stent comprising a surface and a coating disposed on at least a part of the surface, wherein the coating comprises a biologically active material, a first polymeric material, and a physical anti-adhesion agent, wherein the coating has an outer surface and the concentration of the physical anti-adhesion agent is different at the outer surface than the concentration of the physical anti-adhesion agent within the coating and wherein the physical anti-adhesion agent reduces the adhesion of the coating as compared to the same coating without the physical anti-adhesion agent.

24. The stent of claim 23, wherein the concentration of the physical anti-adhesion agent is higher at the outer surface than the concentration of the physical anti-adhesion agent within the coating.
